(19) United States
(12) Patent Application Publication
(10) Pub. No.: US 2002/0173485 A1
(43) Pub. Date: Nov. 21, 2002

(54) Compositions and methods for the treatment or prevention of inflammation

(76) Inventors: Marco Mastradonato, Milan (IT); Gianluca Braguti, Lecco (IT)

Correspondence Address: PENNIE AND EDMONDS, 1155 AVENUE OF THE AMERICAS, NEW YORK, NY, 100362711

(21) Appl. No.: 10/080,624

(22) Filed: Feb. 21, 2002

(30) Foreign Application Priority Data
Jul. 28, 2000 (IT) MI 2000 A 001732

(51) Int. Cl. A61K 31/728; A61K 9/14

(52) U.S. Cl. 514/54; 424/486

(57) ABSTRACT

The present invention relates to compounds containing as active ingredients hyaluronic acid and polyvinylpyrrolidone, for the treatment of inflammatory, ulcerative and painful conditions of moist epithelial surfaces such as mucositis, stomatitis, vestibulitis, aphthous ulcerations, and Behcet's syndrome.

The present application claims priority benefits of International Patent Application No. PCT/EP01/08303 filed Jul. 18, 2001, (published as WO 02/09637 in English on Feb. 7, 2002), which in turn claims priority benefits of Italian Patent Application No. MI 2000 A 001732, filed Jul. 28, 2000, the disclosures of each of which are incorporated herein by reference in their entirety.

### FIELD OF THE INVENTION

This invention relates to certain compositions useful for the management of painful ulcerative and inflammatory conditions of moist surfaces including the mouth, oropharynx, oesophagus, vagina and rectum (including, but not limited to, mucositis, stomatitis, aphthous ulcerations, and Behcet's syndrome).

### BACKGROUND OF THE INVENTION

Aggressive cancer treatment may have toxic effects on normal cells as well as cancer cells. The gastrointestinal tract, including the mouth, is especially affected because these cells are replaced by the body continuously.

Mucositis, an inflammation of the mucous membranes in the mouth, is one of the most common oral problems occurring after chemotherapy and radiation therapy. Mucositis can contribute to oral infections, inability to taste normally and pain arising from the resulting open sores that can develop. Mucositis can become so painful that the patient will not eat or drink, contributing to dehydration and malnutrition.

Radiation therapy to the head and neck for cancers in those areas commonly injure saliva glands and the inside of the mouth which can cause dry mouth, leading to dental disease.

The mucositis problem is not restricted to cancer patients, as mucositis frequently also occurs in HIV patients, particularly when associated with Kaposi's sarcoma, in patients affected with non-Hodgkin's lymphoma, in debilitated elderly patients and in patients receiving BRM treatments like interleukin-2, TNF, interferons, lymphokine-activated lymphocytes and the like.

Such oral problems may make it difficult for the cancer or AIDS patient to receive a complete dose of chemotherapy or radiation therapy. Sometimes treatment must be stopped completely. Such problems are not infrequent: about half of the patients have severe oral lesions that require medical intervention, mostly involving the changes in cancer medication or treatment mentioned above.

Current therapies for mucositis are limited. Cleaning the mouth is recommended to retard the progression of the condition.

Oral cleaning care includes gently cleaning the mouth, moisturizing the lips and mouth, and relieving pain and swelling. A soft toothbrush or toothette cleans teeth well and gently. Cleansing agents can include “salt and soda” (½ tsp. salt and 2 Tbs. of sodium bicarbonate in 32 oz. of warm water), normal saline, sterile water, or sodium bicarbonate (1 tsp. in 8 oz of water). Hydrogen peroxide diluted in equal amounts of water or weak salt water can be used when crusting is present. (This should be used for 1 or 2 days only because it will keep mucositis from healing.) Gentle wiping with a wet gauze dipped in salt water helps remove particles. Toothettes may be too rough for some areas. Particles should be removed before ointments or other medications are put onto the gums or tissues. Rinsing often cleans and moistens the tissues, prevents crusting, and soothes sore gums and tissues. Frequent rinsing prevents particles and bacteria from collecting in the mouth. A salt and baking soda solution neutralizes acids and dissolves thick saliva.

Capsaicin, the active ingredient in hot peppers, reportedly has used to increase a person's ability to tolerate pain. When capsaicin is put on inflamed tissues in the mouth, mucositis pain may decrease as the burning feeling from the capsaicin decreases. Capsaicin is only being used experimentally; however, all side effects are not known.

Mostly, physicians have resorted ice chips or to rather makeshift mixtures of benzocaine with kaopectate and the like. These approaches provide rather limited, temporary relief.

Carrington Laboratories of Irving, Tex. has sold a mucositis product called “Radiacare” for a number of years. However, this product has made limited inroads into the marketplace, and thus has provided few patients relief from the symptoms of mucositis.

Many women get oral aphthous ulceration at specific times of the menstrual cycle and simultaneously get the same kind of ulcers in the genital tract, in particular the vulva and vagina. This is sometimes very severe and can cause retention of urine and require strong painkillers and sedatives. The most severe form is called Behcet's syndrome.

The terms mucositis and stomatitis are often used interchangeably but may include some general distinctions. Mucositis describes a toxic inflammatory reaction affecting the gastrointestinal tract, which may result from exposure to chemotherapeutic agents or ionising radiation. Mucositis typically manifests as an erythematous, burn-like lesion or as random, focal-to-diffuse, ulcerative lesions. Stomatitis refers to an inflammatory reaction affecting the oral mucosa, with or without ulceration, that may be caused or intensified by pharmacological, particularly chemotherapeutic treatments, or by radiotherapy. Stomatitis can range from mild to severe; the patient with severe stomatitis is unable to take anything by mouth.

Thus, there is a clear need for compositions and methods useful for treating or preventing inflammation, including but not limited to, mucositis, stomatitis, aphthous ulcerations, Behcet's syndrome, etc.

Citation of a reference in this or any section of the specification shall not be construed as an admission that such reference is prior art to the present invention.

### SUMMARY OF THE INVENTION

The present invention is directed to a composition comprising from about 0.01 to about 5 percent by weight of hyaluronic acid, or a pharmaceutically acceptable salt thereof, having a molecular weight from about 1.6 and 2.2 million daltons; from about 0.04 to about 15% by weight of a K60 to K100 polyvinylpyrrolidone, or a pharmaceutically acceptable salt thereof; and from about 86 to about 98% water. In one embodiment, the viscosity of the composition is from about 50 to about 500 centipoise. In an embodiment the polyvinylpyrrolidone, or the pharmaceutically acceptable salt thereof, is from about K85 to about K95 and is from about 3 to about 10% by weight of the composition. In another embodiment, the polyvinylpyrrolidone, or the pharmaceutically acceptable salt thereof, is from about 7 to about 10% by weight of the composition. In yet another embodiment, the hyaluronic acid, or the pharmaceutically acceptable salt thereof, is from about 1.8 to about 2.0 million daltons, and from about 0.01 to about 2% by weight of the composition, and wherein the viscosity of the composition is from about 90 to about 1000 centipoise. In yet another embodiment, the hyaluronic acid, or the pharmaceutically acceptable salt thereof, is from about 1.8 to about 2.0 million daltons and from about 0.01% to about 2% by weight of the composition, and wherein the viscosity of the composition is from about 90 to about 1000 centipoise. In a preferred embodiment, the composition is in the form of a gel.

The present invention is also directed to a composition comprising from about 0.04 to about 5 percent by weight of hyaluronic acid, or a pharmaceutically acceptable salt thereof, with a molecular weight from about 1.6 to about 2.2 million daltons; from about 0.08 to about 15% by weight of a K60 to K100 polyvinylpyrrolidone, or a pharmaceutically acceptable salt thereof; and from about 86 to about 98% water. In one embodiment, the viscosity of the composition is from about 50 to about 500 centipoise. In an embodiment, the polyvinylpyrrolidone, or the pharmaceutically acceptable salt thereof, is from about K85 to about K95, and is from about 6 to about 12% by weight of the composition. In another embodiment, the polyvinylpyrrolidone, or the pharmaceutically acceptable salt thereof, is from about 8 to about 10% by weight of the composition. In yet another embodiment, the hyaluronic acid, or the pharmaceutically acceptable salt thereof, is from about 1.8 to about 2.0 million daltons and from about 0.04 to about 2% by weight of the composition. In yet another embodiment, the hyaluronic acid, or the pharmaceutically acceptable salt thereof, is from about 1.8 to about 2.0 million daltons and from about 0.04 to about 2% by weight of the composition. In a preferred embodiment, the composition is in the form of a gel.

The present invention is also directed to a flexible packet comprising a composition comprising from about 0.04 to about 5 percent by weight of hyaluronic acid, or a pharmaceutically acceptable salt thereof, with a molecular weight from about 1.6 to about 2.2 million daltons; from about 0.08 to about 15% by weight of a K60 to K100 polyvinylpyrrolidone, or a pharmaceutically acceptable salt thereof; and from about 86 to about 98% water. In one embodiment, the viscosity of the composition is from about 50 to about 500 centipoise. In a preferred embodiment, the packet is a sealed pouch comprising from about 10 to about 30 milliliters of the composition.

The present invention is also directed to a composition comprising hyaluronic acid, or a pharmaceutically acceptable salt thereof, glycyrrhetinic acid, or a pharmaceutically acceptable salt thereof, and polyvinylpyrrolidone, or a pharmaceutically acceptable salt thereof. In an embodiment, the composition further comprises a viscosity-increasing agent, surfactant, stabilizing agent/preservative, flavor, fragrance, sweetening agent, bioadhesive agent, or a co-solubilizer. The composition may also further comprise a cellulose derivative, acrylic or methacrylic acid polymer or copolymer, ethylene or propylene glycol, polyethoxylated hydrogenated castor oil, EDTA, sodium benzoate, sodium or potassium sorbate, dextrin, sodium saccharin, or aspartame. In yet another embodiment, the composition further comprises an antibacterial agent, disinfectant agent, antifungal agent, analgesic, anti-inflammatory, emollient, or a local anesthetic.

The present invention is also directed to a method for treating or preventing inflammation in a patient comprising administering to a patient in need thereof an effective amount of a composition comprising from about 0.01 to about 5 percent by weight of hyaluronic acid, or a pharmaceutically acceptable salt thereof, having a molecular weight from about 1.6 to about 2.2 million daltons; from about 0.04 to about 15% by weight of a K60 to K100 polyvinylpyrrolidone, or a pharmaceutically acceptable salt thereof; and from about 86 to about 98% water. In one embodiment, the viscosity of the composition is from about 50 to about 500 centipoise. In an embodiment, the composition is administered at least twice daily for at least two consecutive days. In yet another embodiment, the composition is administered at least three times daily for at least four consecutive days.

The present invention is also directed to a method for treating or preventing inflammation in a patient, comprising administering to a patient in need thereof an effective amount of a composition comprising hyaluronic acid, or a pharmaceutically acceptable salt thereof, glycyrrhetinic acid, or a pharmaceutically acceptable salt thereof, and polyvinylpyrrolidone, or a pharmaceutically acceptable salt thereof. In addition to its ordinary meaning, the term treatment encompasses inhibition of progression of symptoms or amelioration of symptoms of inflammation and mucositis.

The present invention can be more fully explained by reference to the following detailed description and illustrative examples.

### DETAILED DESCRIPTION OF THE INVENTION

Surprisingly, the topical administration of a formulation comprising an effective amount of hyaluronic acid, or a pharmaceutically acceptable salt thereof, and polyvinylpyrrolidone, or a pharmaceutically acceptable salt thereof, provides an effective therapeutical or preventive treatment for mucositis and stomatitis of various origin and severity and, more generally, of the lesions of the oro-pharynx cavity and oesophagus, particularly those caused by dental devices and by radio- or chemotherapy.

Without being bound by a particular mode of action, the favorable therapeutic results obtained by the use of the compositions of the present invention are believed to be due to both the interactions between hyaluronic acid, or a pharmaceutically acceptable salt thereof, and polyvinylpyrrolidone, or a pharmaceutically acceptable salt thereof, and the capability of the formulation of adhering to the oral mucosa providing a protective coating for the exposed nerve endings, and thus, reduction of pain and promoting cicatrisation and healing of the lesions. Furthermore, it is believed that the moisturizing effect of the compositions has beneficial effect as it protects mucous membranes from further irritating lesions.

In one embodiment, the present invention involves a composition comprising from about 0.01 to about 5 percent by weight of hyaluronic acid, or a pharmaceutically acceptable salt thereof, having a molecular weight from about 1.6 to about 2.2 million daltons; from about 0.04 to about 15% by weight of a K60 to K100 polyvinylpyrrolidone, or a pharmaceutically acceptable salt thereof, and from about 86 to about 98% water. In one embodiment, the viscosity of the composition is from about 50 to about 500 centipoise.

In an alternative embodiment, the present invention involves a composition comprising from about 0.04 to about 5 percent by weight of hyaluronic acid, or a pharmaceutically acceptable salt thereof, having a molecular weight from about 1.6 to about 2.2 million daltons; from about 0.08 to about 15% by weight of a K60 to K100 polyvinylpyrrolidone, or a pharmaceutically acceptable salt thereof; and from about 86 to about 98% water. In one embodiment, the viscosity of the composition is from about 50 to about 500 centipoise. The compositions of the present invention can be diluted with water, and accordingly, is useful for obtaining the above compositions. In an alternative embodiment, the composition can be diluted with physiological saline.

These compositions can be used by themselves or in admixture with one or more medicaments, excipients and/or adjuvants, preferably forming a viscous and lubricating substance that remains adherent to the surface epithelium. These compositions are suitable for topical administration to epithelial surfaces such as, but not limited to, the oropharynx and oesophagus.

A further aspect of the invention concerns the use of hyaluronic acid, or a pharmaceutically acceptable salt thereof, glycyrrhetinic acid, or a pharmaceutically acceptable salt thereof, and polyvinylpyrrolidone, or a pharmaceutically acceptable salt thereof, for treating or preventing inflammation in a patient. In one embodiment, the inflammation is of epithelial surfaces such as, but not limited to, the oral mucosa, particularly mucositis and stomatitis.

Preferably, the compositions of the present invention are administered by topical application.

The compositions of the invention are preferably in the form of a slightly viscous aqueous liquid (gel) which provides a film-forming and coating effect on the epithelial surfaces such as, but not limited to the oral mucosa.

As explained above, the present invention relates to a composition comprising from about 0.01 to about 5 percent by weight of hyaluronic acid, or a pharmaceutically acceptable salt thereof, having a molecular weight from about 1.6 and 2.2 million daltons; from about 0.04 to about 15% by weight of a K60 to K100 polyvinylpyrrolidone, or a pharmaceutically acceptable salt thereof; and from about 86 to about 98% water. In one embodiment, the viscosity of the composition is from about 50 to about 500 centipoise. In an embodiment, the polyvinylpyrrolidone, or the pharmaceutically acceptable salt thereof, is from about K85 and K95 and is from about 3 and 10% by weight of the composition. Most preferably, the polyvinylpyrrolidone, or the pharmaceutically acceptable salt thereof, is from about 7 to about 10% by weight of the composition. Preferably, the hyaluronic acid, or the pharmaceutically acceptable salt thereof, is from about 1.8 to about 2.0 million daltons and from about 0.01 to about 2% by weight. In one embodiment, the viscosity of the composition is from about 90 to about 1000 centipoise. Preferably, the composition is in the form of a gel. Most preferably, the hyaluronic acid, or the pharmaceutically acceptable salt thereof, is from about 1.8 to about 2.0 million daltons and from about 0.01 to about 2% by weight of the composition, the viscosity of the composition is from about 90 to about 1000 centipoise and the composition is in the form of a gel. Further, glycyrrhetinic acid, or a pharmaceutically acceptable salt thereof, can be present in weight percentages ranging from about 0.01 to about 3% by weight of the composition.

In an embodiment of the present invention, the compositions are provided in a concentrated form for later dilution with water. The compositions comprise from about 0.04 to about 5 percent by weight of hyaluronic acid, or a pharmaceutically acceptable salt thereof, having a molecular weight from about 1.6 to about 2.2 million daltons; from about 0.08 to about 15% by weight of a K60 to K100 polyvinylpyrrolidone, or a pharmaceutically acceptable salt thereof, and from about 86 to about 98% water. In one embodiment, the viscosity of the composition is from about 50 to about 500 centipoise. These compositions preferably comprise polyvinylpyrrolidone, or a pharmaceutically acceptable salt thereof, from about K85 to about K95 and from about 6 to about 12% by weight of the composition, most preferably from about 8 to about 10% by weight of the composition; and comprise hyaluronic acid, or a pharmaceutically acceptable salt thereof, having a molecular weight from about 1.8 to about 2.0 million daltons and from about 0.04 to about 2% by weight of the composition. Preferably, hyaluronic acid, or the pharmaceutically acceptable salt thereof, is from about 1.8 to about 2.0 million daltons in molecular weight and from about 0.04 to about 2% by weight of the composition.

Examples of pharmaceutically acceptable salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The terim “pharmaceutically acceptable salt” also refers to a salt prepared from a compound having an acidic functional group, such as a carboxylic acid or sulfonic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl,N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N, N,-di-lower alkyl-N-(hydroxy lower alkyl)-amines, such as N, N,-dimethyl-N-(2-hydroxyethyl)amine, or tri-(2-hydroxyethyl)amine; N-methyl-D-glucamine; and amino acids such as arginine, lysine, and the like.

The compositions of the present inventions can comprise a pharmaceutically acceptable excipient, preferably for topical administration, such as one or more of the following:

viscosity-increasing agent;

surfactant;

stabilizing agent/preservative;

flavor, fragrance, sweetening agent;

bioadhesive;

co-solubilizer.

Examples of said excipients comprise cellulose derivatives, acrylic or methacrylic acids polymers or copolymers, ethylene or propylene glycols, polyethoxylated hydrogenated castor oil, EDTA, sodium benzoate, sodium or potassium sorbate, dextrins, sodium saccharin, aspartame and other excipients conventionally used in the formulation of collutories or liquid oral forms. Oral formulation can include standard carriers such as pharmaceutical grades of mannitol, lactose, starch, magnesium stearate, sodium saccharine, cellulose, magnesium carbonate, etc. Additional examples of suitable excipients are described in “Remington's Pharmaceutical Sciences” by E. W. Martin.

The compositions of the present invention may further comprise one or more other active ingredients, such as an antibacterial, disinfectant, antifungal, analgesic, other anti-inflammatory, emollients, local anaesthetics and the like. Suitable antimicrobials include, but are not limited to, quaternary ammonium salts such as benzalkonium chloride.

The precise dose to be employed in the composition will depend on the route of administration, and the seriousness of the disease or disorder, and should be decided according to the judgment of the practitioner and each patient's circumstances. In principle, however, for oral applications, a wash or gargle with 10-50 ml of solution, optionally diluted in water, for a time of about up to two or three minutes at least two but preferably three times or more daily, most preferably before meals, will be sufficient to provide an optimal therapeutic or preventive response. The treatment can be protracted until remission of symptoms, usually for at least 2 days, but preferably 5-10 days. More prolonged treatments are not contraindicated, considering the low, if any, toxicity of the components of the formulations of the invention.

The present invention also provides a pharmaceutical pack or kit comprising one or more containers, e.g., a flexible packet, vial, ampoule, bottle and the like, filled with one or more of the ingredients of the compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a preferred embodiment, the compositions of the present invention can be presented as single- or multi-dose forms in a flexible packet. Preferably, the compositions of the present invention are packaged in the concentrated form in flexible packets with a dose of from about 10 to about 30 ml per packet that can be diluted with water to create about 40-60 ml of product for use by the patient.

The following series of examples are presented by way of illustration and not by way of limitation on the scope of the invention.

### EXAMPLE 1

Qualitative-quantitative composition percent composition:

| Ingredient | % By Weight |
|---|---|
| Sodium hyaluronate | 0.1 |
| Glycyrrhetinic acid | 0.06 |
| PVP (K60 to K100) | 9.0 |
| Maltodextrin | 6.00 |
| Propylene glycol | 2.94 |
| Potassium sorbate | 0.3 |
| Sodium benzoate | 0.3 |
| Hydroxyethyl cellulose | 1.5 |
| Hydrogenated castor oil PEG-40 | 0.27 |
| Disodium EDTA | 0.1 |
| Benzalkonium chloride | 0.5 |
| Perfume (Glycyrrhiza Comp. 2717) | 0.16 |
| Sodium saccharin | 0.1 |
| Depurated water | 78.44 |

To prepare this composition, water was placed in a turboemulsifier, then a mixture of potassium sorbate, sodium benzoate and disodium EDTA was added, followed by hyaluronic acid and maltodextrin. The mixture was stirred after each addition until complete dissolution of the components. After that, PVP was slowly added under stirring and vacuum (30 mm Hg) until complete solvation. Then sodium saccharin and hydroxyethylcellulose were subsequently added, the whole was subjected to vacuum and left under stirring until complete salvation. Afterwards, hydrogenated castor oil 40/OE and perfume, benzalkonium chloride, and a mixture of propylene glycol and glycyrrhetinic acid were added in that order, stirring after each addition until complete dissolution of the components. When the additions were completed, the mixture was stirred under vacuum for 30 minutes.

For a concentrated version of the invention, 10 ml or 15 ml of the above composition were distributed in a packet or mono-dose vial, which can be diluted with 30-50 ml of water before use; for the ready-to-use version, the composition disclosed above was diluted with depurated water to a concentration of 50%, and 200 ml or 300 ml of the resulting composition were distributed in bottles.

### EXAMPLE 2

### IN VIVO DATA

Thirty patients, of age range from 30 to 60 years, were evaluated, 10 of them were AIDS patients 30 to 40 years of age who were also receiving anti-retroviral therapy. All patients in the study were affected with inflammatory pathologies of the oral cavity of various aetiology:

12 cases of oro-pharyngeal mucositis;

4 cases of aphthous lesions of the oral cavity;

4 cases of post-traumatic lesions;

3 cases of Lichen Planus of the oral cavity;

3 cases of radiotherapy-induced stomatitis;

3 cases of oral cavity surgery side effects; and

1 case of leukoplakia.

Patients were treated with the composition described in Example 1 in 15 ml sachets (packets) diluted in water in a 1:4 ratio. The slightly viscous solution was retained in the mouth for 2-3 minutes during which it was gargled and swirled about to obtain homogeneous distribution on the whole surface of the oral mucosa. The solution was then discharged. The patients refrained from eating or drinking for about at least 15 minutes after gargling.

The formulation was used three times a day 60 minutes before meal times for seven consecutive days.

At the end of the treatment, the extent of inflammation and lesions, the decrease or disappearance of dysphagia for solid and semi-solid foods, and liquids, and the duration of the activity of the product were evaluated.

After the first administration, more than 80% of patients perceived within a few hours reduction of pain so as to permit food intake. The effect lasted three or four hours.

Healing of the lesions of the oral mucosa occurred after 3-4 days of treatment in about 60% of treated cases. The percentage reached 90% at the end of one week of treatment. In the remaining three cases only a pathological condition persisted, but with improved symptoms compared with the beginning of the treatment, providing a remarkable improvement of life quality and restoring a normal, differentiated diet.

### EXAMPLE 3

Two patients with throat pain (sore throat) were unable to obtain relief with analgesics or other topical agents. Patients were treated with the composition described in Example 1 in 15 ml packets, the contents of which were diluted in water in a 1:4 ratio. The solution was retained in the mouth for about one minute during which time it was gargled to obtain good contact with the tissues of the throat. The solution was then discharged. Within ten minutes, the patients experienced dramatic relief of their sore throat symptoms, which relief persisted for several hours.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

Various publications are cited herein, the disclosures of which are incorporated by reference in their entireties.

What is claimed is:

1. A composition, comprising:
from about 0.01 to about 5 percent by weight of hyaluronic acid, or a pharmaceutically acceptable salt thereof, having a molecular weight from about 1.6 and 2.2 million daltons;
from about 0.04 to about 15% by weight of a K60 to K100 polyvinylpyrrolidone, or a pharmaceutically acceptable salt thereof; and
from about 86 to about 98% water,
wherein the viscosity of the composition is from about 50 to about 500 centipoise.

2. The composition of claim 1, wherein the polyvinylpyrrolidone, or the pharmaceutically acceptable salt thereof, is from about K85 to about K95 and is from about 3 to about 10% by weight of the composition.

3. The composition of claim 2, wherein the polyvinylpyrrolidone, or the pharmaceutically acceptable salt thereof, is from about 7 to about 10% by weight of the composition.

4. The composition of claim 1, wherein the hyaluronic acid, or the pharmaceutically acceptable salt thereof, is from about 1.8 to about 2.0 million daltons, and from about 0.01 to about 2% by weight of the composition, and wherein the viscosity of the composition is from about 90 to about 1000 centipoise.

5. The composition of claim 4, in the form of a gel.

6. The composition of claim 3, wherein the hyaluronic acid, or the pharmaceutically acceptable salt thereof, is from about 1.8 to about 2.0 million daltons and from about 0.01% to about 2% by weight of the composition, and wherein the viscosity of the composition is from about 90 to about 1000 centipoise.

7. The composition of claim 6, in the form of a gel.

8. A composition comprising:
from about 0.04 to about 5 percent by weight of hyaluronic acid, or a pharmaceutically acceptable salt thereof, with a molecular weight from about 1.6 to about 2.2 million daltons;
from about 0.08 to about 15% by weight of a K60 to K100 polyvinylpyrrolidone, or a pharmaceutically acceptable salt thereof, and
from about 86 to about 98% water,
wherein the viscosity of the composition is from about 50 to about 500 centipoise.

9. The composition of claim 8, wherein the polyvinylpyrrolidone, or the pharmaceutically acceptable salt thereof, is from about K85 to about K95, and is from about 6 to about 12% by weight of the composition.

10. The composition of claim 9, wherein the polyvinylpyrrolidone, or the pharmaceutically acceptable salt thereof, is from about 8 to about 10% by weight of the composition.

11. The composition of claim 8, wherein the hyaluronic acid, or the pharmaceutically acceptable salt thereof, is from about 1.8 to about 2.0 million daltons and from about 0.04 to about 2% by weight of the composition.

12. The composition of claim 11, in the form of a gel.

13. The composition of claim 10, wherein the hyaluronic acid, or the pharmaceutically acceptable salt thereof, is from about 1.8 to about 2.0 million daltons and from about 0.04 to about 2% by weight of the composition.

14. The composition of claim 13, in the form of a gel.

15. A flexible packet comprising the composition of claim 8.

16. The packet of claim 15, being a sealed pouch comprising from about 10 to about 30 milliliters of the composition.

17. A composition comprising hyaluronic acid or a pharmaceutically acceptable salt thereof; glycyrrhetinic acid or a pharmaceutically acceptable salt thereof; and polyvinylpyrrolidone or a pharmaceutically acceptable salt thereof.

18. The composition of claim 17, further comprising a viscosity-increasing agent, surfactant, stabilizing agent/preservative, flavour, fragrance, sweetening agent, bioadhesive agent, or a co-solubilizer.

19. The composition of claim 18, further comprising a cellulose derivative, acrylic or methacrylic acid polymer or copolymer, ethylene or propylene glycol, polyethoxylated hydrogenated castor oil, EDTA, sodium benzoate, sodium or potassium sorbate, dextrin, sodium saccharin, or aspartame.

20. The composition of claim 17, further comprising an antibacterial agent, disinfectant agent, antifungal agent, analgesic, anti-inflammatory, emollient, or a local anesthetic.

21. A method for treating or preventing inflammation in a patient comprising:
administering to a patient in need thereof an effective amount of a composition comprising:
(i) from about 0.01 to about 5 percent by weight of hyaluronic acid, or a pharmaceutically acceptable salt thereof, having a molecular weight from about 1.6 to about 2.2 million daltons;
(ii) from about 0.04 to about 15% by weight of a K60 to K100 polyvinylpyrrolidone, or a pharmaceutically acceptable salt thereof; and
(iii) from about 86 to about 98% water,
wherein the viscosity of the composition is from about 50 to about 500 centipoise.

22. The method of claim 21, wherein the composition is administered at least twice daily for at least two consecutive days.

23. The method of claim 21, wherein the composition is administered at least three times daily for at least four consecutive days.

24. A method for treating or preventing inflammation in a patient, comprising administering to a patient in need thereof an effective amount of a composition comprising hyaluronic acid or a pharmaceutically acceptable salt thereof; glycyrrhetinic acid or a pharmaceutically acceptable salt thereof; and polyvinylpyrrolidone or a pharmaceutically acceptable salt thereof.

25. The method of claim 21, wherein the administration is by topical application.

26. The method of claim 24, wherein the administration is by topical application.
